(19) United States
(12) Patent Application Publication
(10) Pub. No.: US 2011/0313004 A1
(43) Pub. Date: Dec. 22, 2011

(54) DEUTERATED PYRIDINONES

(75) Inventor: Scott L. Harbeson, Cambridge, MA (US)

(73) Assignee: CONCERT PHARMACEUTICALS, INC., Lexington, MA (US)

(21) Appl. No.: 13/132,784

(22) PCT Filed: Dec. 3, 2009
(86) PCT No.: PCT/US2009/066606

§ 371 Date: Sep. 9, 2011

Related U.S. Application Data
(60) Provisional application No. 61/200,849, filed on Dec. 4, 2008.

(51) Int. Cl. A61K 31/4412; A61P 11/00; A61P 25/00; A61P 13/12; A61P 9/00; A61P 17/02; A61P 25/28; A61P 1/16; A61P 15/00; A61P 35/00; A61P 37/06; A61P 17/00; C07D 213/64; A61P 43/00

(52) U.S. Cl. 514/345; 546/301

(57) ABSTRACT

This invention relates to novel substituted pyridinones, their deuterium-modified derivatives and pharmaceutically acceptable salts thereof. This invention also provides compositions comprising a compound of this invention and the use of such compositions in methods of treating diseases and conditions that are beneficially treated by administering a TNF (tumor necrosis factor) alpha production inhibitor/TGF (transforming growth factor) beta inhibitor.

### RELATED APPLICATION

This application claims the benefit of U.S. Provisional Application No. 61/200,849, filed on Dec. 4, 2008. The entire teachings of the above application are incorporated herein by reference.

### BACKGROUND OF THE INVENTION

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

Pirfenidone, also known as 5-methyl-1-phenylpyridin-2(1H)-one, is thought to inhibit collagen synthesis, down-regulate e cytokine production, and block fibroblast proliferation and stimulation in response to cytokines.

Pirfenidone is currently pre-registered for idiopathic pulmonary fibrosis (Japan), and is in clinical trials for idiopathic pulmonary fibrosis (Europe and US), neurofibromatosis, Hermansky-Pudlak syndrome, diabetic nephropathy, renal failure, hypertrophic cardiomyopathy (HCM), glomerulosclerosis (FSGS), radiation-induced fibrosis, multiple sclerosis, and uterine leiomyomas (fibroids).

Adverse events experienced by patients dosed with pirfenidone include, but are not limited to, nausea, gastrointestinal disturbances, fatigue, headache, photosensitive skin rash, and moderate photosensitivity (Raghu, G et al., Am J Resp Crit Care Med, 1999, 159(4):1061. Thus, despite the beneficial activities of pirfenidone, there is a continuing need for new compounds to treat the aforementioned diseases and conditions.

### SUMMARY OF THE INVENTION

This invention relates to novel substituted pyridinones, their derivatives and pharmaceutically acceptable salts thereof. This invention also provides compositions comprising a compound of this invention and the use of such compositions in methods of treating diseases and conditions that are beneficially treated by administering a TNF (tumor necrosis factor)-alpha production inhibitor/TGF (transforming growth factor)-beta inhibitor.

### DETAILED DESCRIPTION OF THE INVENTION

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

The term “alkyl” refers to a monovalent saturated hydrocarbon group. C₁-C₃ alkyl is an alkyl having from 1 to 3 carbon atoms. An alkyl may be linear or branched. Examples of alkyl groups include methyl; ethyl; and propyl, including n-propyl and isopropyl.

It will be recognized that some variation of natural isotopic abundance occurs in a synthesized compound depending upon the origin of chemical materials used in the synthesis. Thus, a preparation of pirfenidone will inherently contain small amounts of deuterated isotopologues. The concentration of naturally abundant stable hydrogen isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In a compound of this invention, when a particular position is designated as “D” or “deuterium”, it is understood that the abundance of deuterium at that position is at least 3340 times greater than the natural abundance of deuterium, which is 0.015% (i.e., at least 50.1% incorporation of deuterium at that site). In this application the ratio between the isotopic abundance and the natural abundance of a specified isotope is termed “isotopic enrichment factor”.

When a position is designated specifically as “H” or “hydrogen”, the position is understood to have hydrogen at its natural abundance isotopic composition.

In the compounds of this invention any atom not specifically designated as a particular isotope is meant to represent any stable isotope of that atom.

In certain embodiments, each designated deuterium atom in a compound of this invention has an isotopic enrichment factor of at least 3340 (50.1% deuterium incorporation at each designated deuterium atom), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” when referring to the compounds of the invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium, “Stereoisomer” refers to both enantiomers and diastereomers, “Tert”, “^{t}”, and “t-” each refer to tertiary. “US” refers to the United States of America.

The term “substituted with deuterium” means that one or more hydrogen atoms in the indicated moiety are substituted with a deuterium atom.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

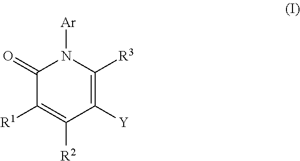

or a pharmaceutically acceptable salt thereof, wherein:

Ar is

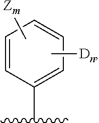

wherein:

Z, if present, is selected from halo, —OH, —CH₃, —CD₃, —OCH₃, —OCD₃, —CF₃, and —NO₂;

m is 0 or 1;

n is 0 or an integer from 1 to 5;

m+n≦5;

R¹ is hydrogen, deuterium, or a group selected from phenyl, chlorophenyl, and C₁-C₃ alkyl, which group is optionally substituted with one or more deuterium atoms;

each of R² and R³ is independently hydrogen or deuterium; and

Y is selected from —CD₃, —CD₂CD₃, —(CD₂)₂CD₃, —C(O)D, —C(O)CD₃, —CD₂F, —CDF₂, —CD₂OCH₃, —CD₂OCD₃, —CH₂OCD₃, —CD₂OH, and —CF₃;

provided that at least one of R¹, R², R³, Y, Z and Ar is or contains deuterium; and

provided that when R¹ is hydrogen or deuterium, and Y is —CD₃, then m is 1.

In one embodiment of a compound of Formula I, n is 0. In another embodiment, n is 4 and m is 1. In another embodiment, n is 5 and m is 0.

Another embodiment provides a compound of Formula I, wherein m is 1 and the compound has the Formula Ia:

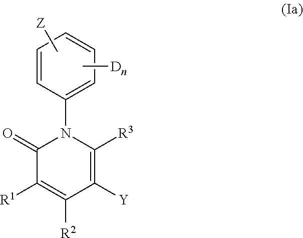

wherein R¹, R², R³, Z, and Y are as described above; and n is 0, 1, 2, 3, or 4. In one aspect of this embodiment, n is 0 or 4.

In one embodiment of a compound of Formula I or Ia, Z, if present, is selected from —OH, —CH₃, —CD₃, —CF₃, and —NO₂.

A more specific embodiment of a compound of Formula Ia, is a compound of Formula Ib:

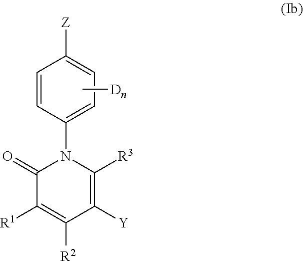

wherein R¹, R², R³, Z, and Y are as described above and n is 0, 1, 2, 3, or 4. In one aspect of this embodiment, n is 0 or 4.

Another embodiment of this invention provides a compound of Formula I, Ia or Ib, wherein R¹ is selected from hydrogen, deuterium, —CH₃, —CD₃, —CD₂CH₃, —CD₂CD₃, and —CH₂CD₃. In another aspect, R¹ is hydrogen or deuterium.

In another embodiment of Formula I, Ia or Ib, R² is the same as R³. In one aspect of this embodiment, each of R¹, R², and R³ is hydrogen.

Another embodiment provides a compound of Formula I, Ia or Ib wherein Y is selected from —CF₃, —CD₃, —CD₂CD₃, —(CD₂)₂CD₃, —CD₂F, —CDF₂, —CD₂OCH₃, —CD₂OCD₃, —CH₂OCD₃, and —CD₂OH. In one aspect of this embodiment, each of R¹, R², and R³ is hydrogen.

The present invention also provides a compound of Formula II

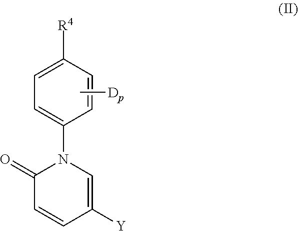

wherein Y is selected from —CF₃, —CD₃, —CD₂CD₃, —(CD₂)₂CD₃, —CDF₂, —CD₂OCH₃, —CD₂OCD₃, —CH₂OCD₃, and —CD₂OH;

R⁴ is selected from hydrogen, deuterium, fluorine, chlorine, —OH, —CH₃, —CD₃, —OCH₃, —OCD₃, and —CF₃;

p is 0 or an integer from 1 to 4 (e.g., 1, 2, 3 or 4);

when Y is —CF₃, R⁴ is selected from —CD₃, and —OCD₃; and

when Y is —CD₃, R⁴ is not hydrogen or deuterium.

Examples of specific compounds of Formula II, wherein p is 0, are set forth in Table 1 below.

TABLE 1
Exemplary Compounds of Formula II.

| Compound | R⁴ | Y |
|---|---|---|
| 101 | —H | —CD₂OH |
| 102 | —H | —CD₂OCH₃ |
| 103 | —H | —CD₂OCD₃ |
| 104 | —OH | —CD₃ |
| 105 | —OH | —CD₂OH |
| 106 | —OH | —CD₂OCH₃ |
| 107 | —OH | —CD₂OCD₃ |
| 108 | —F | —CD₃ |
| 109 | —F | —CD₂OH |
| 110 | —F | —CD₂OCH₃ |
| 111 | —F | —CD₂OCD₃ |
| 112 | —CH₃ | —CD₃ |
| 113 | —CH₃ | —CD₂OH |
| 114 | —CH₃ | —CD₂OCH₃ |
| 115 | —CH₃ | —CD₂OCD₃ |
| 116 | —OCH₃ | —CD₃ |
| 117 | —OCH₃ | —CD₂OH |
| 118 | —OCH₃ | —CD₂OCH₃ |
| 119 | —OCH₃ | —CD₂OCD₃ |
| 120 | —CD₃ | —CD₃ |
| 121 | —CD₃ | —CD₂OH |
| 122 | —CD₃ | —CD₂OCH₃ |
| 123 | —CD₃ | —CD₂OCD₃ |
| 124 | —CD₃ | —CF₃ |
| 125 | —OCD₃ | —CD₃ |
| 126 | —OCD₃ | —CD₂OH |
| 127 | —OCD₃ | —CD₂OCH₃ |
| 128 | —OCD₃ | —CD₂OCD₃ |
| 129 | —OCD₃ | —CF₃. |

In one embodiment of the compound of Formula II, R⁴ is selected from hydrogen, deuterium, —OH, —CH₃, —CD₃, and —CF₃. In an example of this embodiment, the compound is selected from the group consisting of the compounds set forth in the table below wherein p is 0:

| Compound | R⁴ | Y |
|---|---|---|
| 101 | H | —CD₂OH |
| 102 | H | —CD₂OCH₃ |
| 103 | H | —CD₂OCD₃ |
| 104 | OH | —CD₃ |
| 105 | OH | —CD₂OH |
| 106 | OH | —CD₂OCH₃ |
| 107 | OH | —CD₂OCD₃ |
| 112 | CH₃ | —CD₃ |
| 113 | CH₃ | —CD₂OH |
| 114 | CH₃ | —CD₂OCH₃ |
| 115 | CH₃ | —CD₂OCD₃ |
| 120 | CD₃ | —CD₃ |
| 121 | CD₃ | —CD₂OH |
| 122 | CD₃ | —CD₂OCH₃ |
| 123 | CD₃ | —CD₂OCD₃ |
| 124 | CD₃ | —CF₃. |

Another embodiment of Formula II provides a compound wherein each of R¹, R², and R³ is hydrogen, the compound represented by Formula IIa:

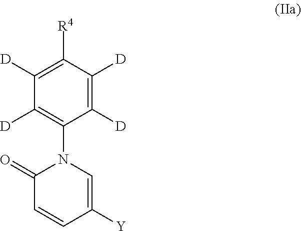

wherein:

Y is selected from —CF₃, —CD₃, —CD₂CD₃, —(CD₂)₂CD₃, —CD₂F, —CDF₂, —CD₂OCH₃, —CD₂OCD₃, —CH₂OCD₃, and —CD₂OH; and

R⁴ is selected from hydrogen, deuterium, fluorine, chlorine, —OH, —CH₃, —CD₃, —OCH₃, —OCD₃, and —CF₃;

when Y is —CF₃, R⁴ is selected from —CD₃ and —OCD₃; and

when Y is —CD₃, R⁴ is not hydrogen or deuterium.

Examples of specific compounds of Formula IIa are set in Table 2 below.

TABLE 2
Exemplary Compounds of Formula IIa

| Compound | R⁴ | Y |
|---|---|---|
| 131 | -D | —CD₂OH |
| 132 | -D | —CD₂OCH₃ |
| 133 | -D | —CD₂OCD₃ |
| 134 | —OH | —CD₃ |
| 135 | —OH | —CD₂OH |
| 136 | —OH | —CD₂OCH₃ |
| 137 | —OH | —CD₂OCD₃ |
| 138 | —F | —CD₃ |
| 139 | —F | —CD₂OH |
| 140 | —F | —CD₂OCH₃ |
| 141 | —F | —CD₂OCD₃ |
| 142 | —CH₃ | —CD₃ |
| 143 | —CH₃ | —CD₂OH |
| 144 | —CH₃ | —CD₂OCH₃ |
| 145 | —CH₃ | —CD₂OCD₃ |
| 146 | —OCH₃ | —CD₃ |
| 147 | —OCH₃ | —CD₂OH |
| 148 | —OCH₃ | —CD₂OCH₃ |
| 149 | —OCH₃ | —CD₂OCD₃ |
| 150 | —CD₃ | —CD₃ |
| 151 | —CD₃ | —CD₂OH |
| 152 | —CD₃ | —CD₂OCH₃ |
| 153 | —CD₃ | —CD₂OCD₃ |
| 154 | —CD₃ | —CF₃ |
| 155 | —OCD₃ | —CD₃ |
| 156 | —OCD₃ | —CD₂OH |
| 157 | —OCD₃ | —CD₂OCH₃ |
| 158 | —OCD₃ | —CD₂OCD₃ |
| 159 | —OCD₃ | —CF₃ |
| 160 | —Cl | —CD₃ |
| 161 | —Cl | —CD₂OH |
| 162 | —Cl | —CD₂OCH₃ |
| 163 | —Cl | —CD₂OCD₃. |

In one embodiment of the compound of Formula I or of Formula IIa, the compound does not comprise compounds 139, 156, and 161.

In one embodiment of the compound of Formula IIa, R⁴ is selected from hydrogen, deuterium, —OH, —CD₃, and —CF₃. In an example of this embodiment, the compound is selected from the group consisting of the compounds set forth in the table below:

| Compound | R⁴ | Y |
|---|---|---|
| 131 | D | —CD₂OH |
| 132 | D | —CD₂OCH₃ |
| 133 | D | —CD₂OCD₃ |
| 134 | OH | —CD₃ |
| 135 | OH | —CD₂OH |
| 136 | OH | —CD₂OCH₃ |
| 137 | OH | —CD₂OCD₃ |
| 142 | CH₃ | —CD₃ |
| 143 | CH₃ | —CD₂OH |
| 144 | CH₃ | —CD₂OCH₃ |
| 145 | CH₃ | —CD₂OCD₃ |
| 150 | CD₃ | —CD₃ |
| 151 | CD₃ | —CD₂OH |
| 152 | CD₃ | —CD₂OCH₃ |
| 153 | CD₃ | —CD₂OCD₃ |
| 154 | CD₃ | —CF₃. |

In another set of embodiments, any atom not designated as deuterium in any of the embodiments of Formula I, Ia, Ib, II, or IIa set forth above is present at its natural isotopic abundance.

The synthesis of compounds of Formula I, Ia, Ib, II or IIa can be readily achieved by synthetic chemists of ordinary skill by reference to the Exemplary Synthesis and Examples disclosed herein. Relevant procedures and intermediates are disclosed, for instance in Castaner, J et al., Drugs Fut, 1977, 2(6):396; Chinese Patent Application Nos. CN 1817862, and CN 1386737; and PCT Patent publication No. WO 2003014087.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

Exemplary Synthesis

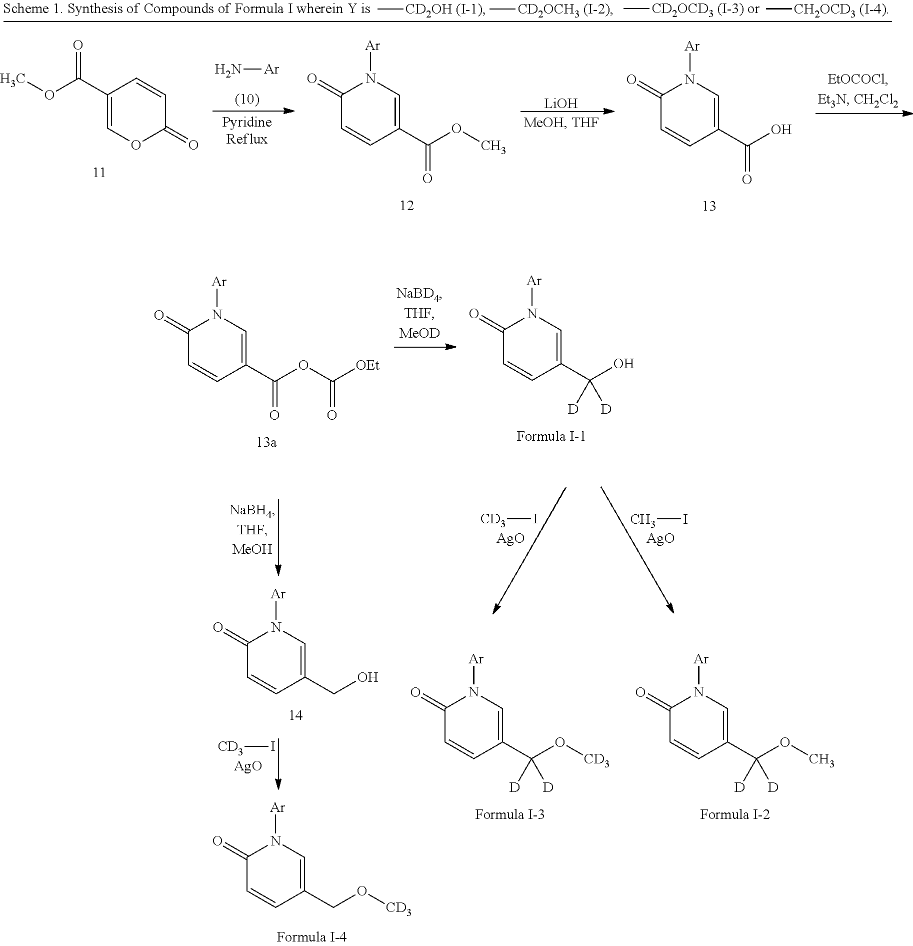

Scheme 1 provides a general method for synthesizing compounds of Formula I, wherein Y is —CD₂OH, —CD₂OCH₃, —CD₂OCD₃ or —CH₂OCD₃. Appropriately deuterated, optionally substituted aniline 10 undergoes reaction with methyl coumalate 11 to provide ester 12. The ester 12 is then hydrolyzed to acid 13, which is subsequently reduced via the intermediate mixed anhydride 13a with either NaBH₄ or with NaBD₄ to produce compound 14 or a compound of Formula I-1. Alcohol 14 can be converted to a compound of Formula I-4 (wherein Y is —CH₂OCD₃) by reaction with d₃-methyl iodide. A compound of Formula I-1 can be converted to a compound of Formula I-2 (wherein R² is —CD₂OCH₃) or a compound of Formula I-3 (wherein R² is —CD₂OCD₃) by reaction with methyl iodide or d₃-methyl iodide, respectively.

Undeuterated and deuterated anilines 10 that can be used in Scheme 1 include the following commercially available compounds: aniline, 4-aminophenol, 4-fluoroaniline, 4-methylaniline, 4-methoxyaniline, 4-(d₃-methyl)aniline, 2,3,4,5,6-d₅ aniline, 4-amino-2,3,5,6-d₄-phenol, and 2,3,5,6-d₄-4-(d₃-methyl)aniline.

The treatment of commercially available 2,3,5,6-d₄-4-fluorobenzoic acid with sulfuric acid, sodium azide and chloroform according to the method disclosed in Repine, J T et al., Tet Lett, 2007, 48(31):4439-4441 produces 2,3,5,6-d₄-4-fluoroaniline:

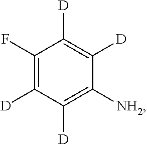

which may also be used as provided in Scheme 1.

Another deuterated aniline 10 that may be used according to Scheme 1 is 2,3,5,6-d₄-4-methylaniline:

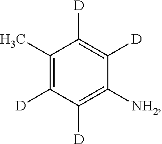

which is prepared according to the procedure described by Frischkorn C G et al., J Label Comp Radiopharm, 1978, 14(4):507-513.

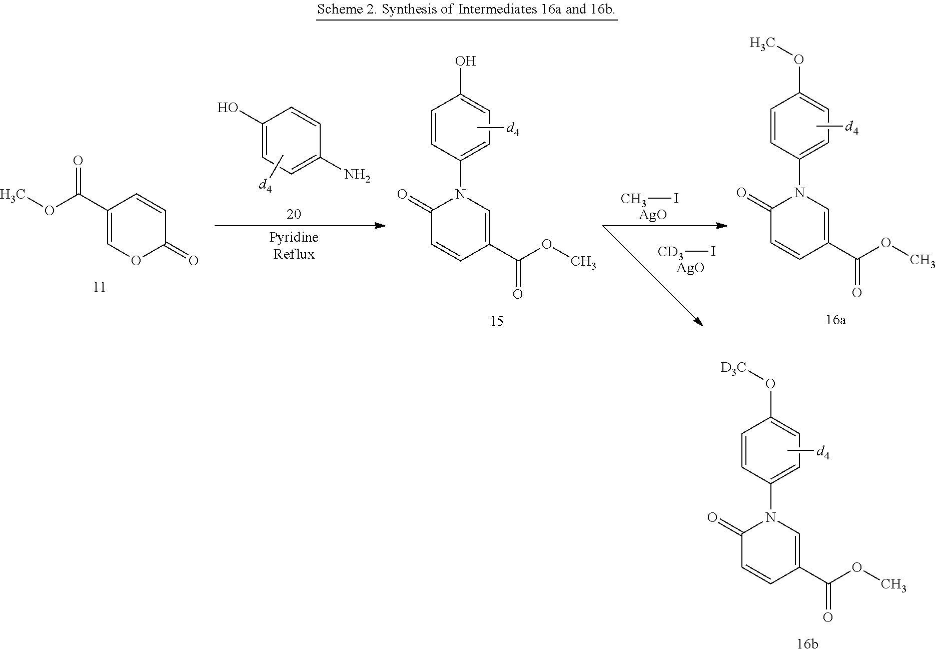

Scheme 2 depicts the synthesis of deuterated intermediates 16a or 16b, which can be used in Scheme 1 as deuterated variants of ester 12. Phenol 20 undergoes reaction with methyl coumalate 11 in refluxing pyridine to provide 15. Compound 15 can then be converted to methyl ether 16a or 16b by reaction with neat methyl iodide or d₃-methyl iodide, respectively, in the presence of AgO.

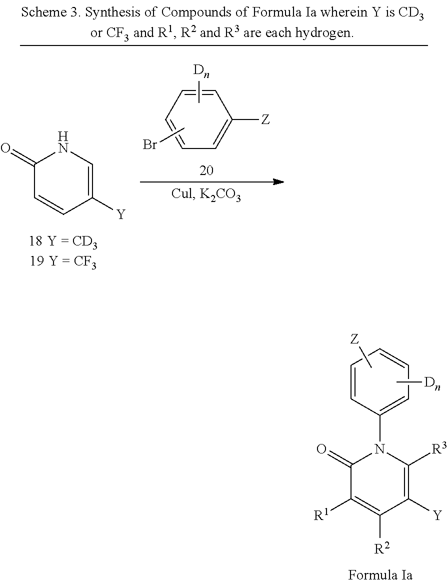

Scheme 3 depicts the synthesis of compounds of Formula Ia wherein Y is CD₃ or CF₃. A Buchwald reaction is used to couple pyridinone 18 (Y is CD₃) or 19 (Y is CF₃; commercially available) with the appropriate arylbromide 20 to provide a compound of Formula Ia. Commercially available aryl bromides 20 contemplated for use in Scheme 3 include bromobenzene, 4-bromophenol, 1-bromo-4-fluorobenzene, 4-bromotoluene, 4-bromoanisole, 1-bromo-4-(d₃ methyl)benzene, 1-bromo-2,3,4,5,6-d₅ benzene, 4-bromo-2,3,5,6-d₄ phenol, 1-bromo-2,3,5,6-d₄-4-(d₃ methyl)benzene.

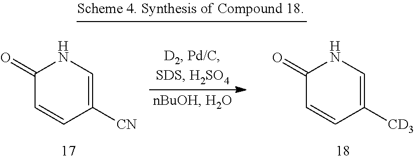

The synthesis of compound 18 is depicted in Scheme 4. Commercially available 6-oxo-1,6-dihydropyridine-3-carbonitrile 17 is dissolved in sodium dodecyl sulfate (“SDS”) and sulfuric acid in n-butanol/water and is then hydrogenated with deuterium gas over palladium on carbon to produce 5-(methyl-d₃)-pyridin-2(1H)-one 18.

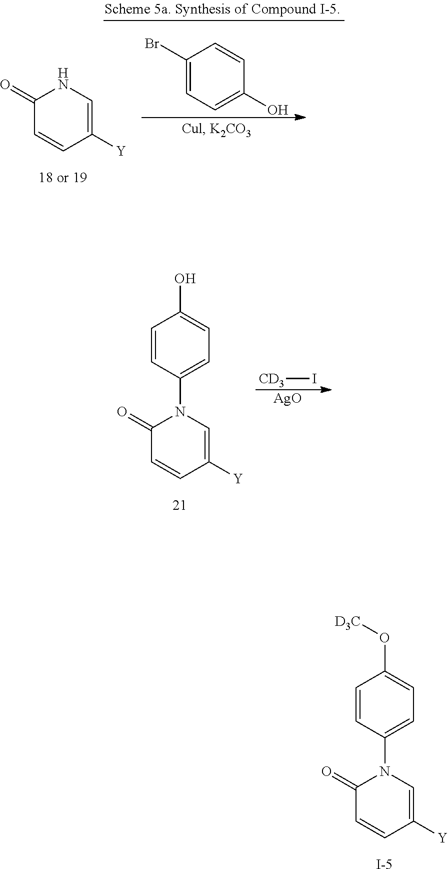

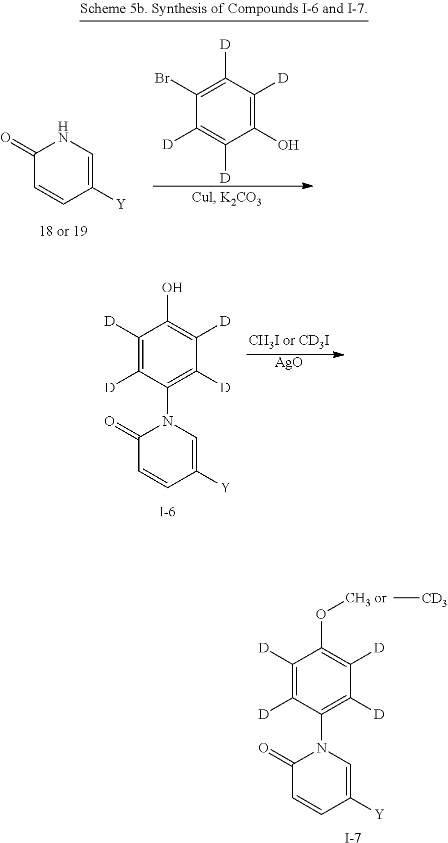

Compounds I-5, I-6, and I-7 can be accessed as shown in Schemes 5a and 5b. For compound I-5, Buchwald coupling of bromophenol 20 to 2-pyridinone 18 or 19 provides the pyridinone phenol 21. Phenol 21 can then be treated with methyl iodide or d₃-methyl iodide to provide compound I-5. Similarly, coupling of d4-bromophenol 20 to 2-pyridinone 18 or 19 provides a compound I-6 wherein Z is —OH, which subsequently can undergo reaction with methyl iodide or d₃-methyl iodide to provide a compound I-7.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I (e.g., including any of the formulae herein), or a pharmaceutically acceptable salt, solvate, or hydrate of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed, Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as pirfenidone. Such agents include those indicated as being useful in combination with pirfenidone, including but not limited to, those described in WO 2004019863, WO 2004105684, WO 2005013917, WO 2005038056, and WO 2005110478.

Preferably, the second therapeutic agent is useful in the treatment of a patient suffering from or susceptible to a disease or condition selected from Such diseases include, but are not limited to, idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis, including secondary progressive multiple sclerosis; uterine leiomyomas (fibroids); alcoholic liver disease including hepatic steatosis, hepatic fibrosis and hepatic cirrhosis; keloid scarring; hepatitis C virus (HCV) infection; proliferative disorders, including angiogenesis-mediated disorders, cancer (including glioma, glioblastoma, breast cancer, colon cancer, melanoma and pancreatic cancer) and fibrotic disorders; interstitial lung diseases; atrial fibrillation (AF); organ transplant rejection; and scleroderma and related fibrotic conditions of the skin.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 2 to about 8000 mg per treatment. In more specific embodiments the range is from about 20 to 4000 mg or from 40 to 1600 mg or most specifically from about 200 to 800 mg per treatment. Treatment typically is administered one to three times daily. In another embodiment, an effective amount of a compound of this invention is between about 800 to 2400 mg/day.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pirfenidone.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the production and activity of TNF-alpha and TGF-beta in a cell, comprising contacting a cell with one or more compounds of Formula I, Ia, Ib, II or IIa or a pharmaceutically acceptable salt therein.

According to another embodiment, the invention provides a method of treating a disease that is beneficially treated by pirfenidone in a patient in need thereof comprising the step of administering to said patient an effective amount of a compound or a composition of this invention. Such diseases are well known in the art and are disclosed in, but not limited to the following patents and published applications: WO 2001058448, WO 2003051388, WO 2004019863, WO 2004073713, WO 2004105684, WO 2005039598, WO 2005038056, WO 2005110478, and WO 2007053610.

Such diseases include, but are not limited to, idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; focal segmental glomerulosclerosis (FSGS); radiation-induced fibrosis; multiple sclerosis, including secondary progressive multiple sclerosis; uterine leiomyomas (fibroids); alcoholic liver disease including hepatic steatosis, hepatic fibrosis and hepatic cirrhosis; keloid scarring; hepatitis C virus (HCV) infection; proliferative disorders, including angiogenesis-mediated disorders, cancer (including glioma, glioblastoma, breast cancer, colon cancer, melanoma and pancreatic cancer) and fibrotic disorders; interstitial lung diseases; atrial fibrillation (AF); organ transplant rejection; and scleroderma and related fibrotic conditions of the skin.

In one particular embodiment, the method of this invention is used to treat a disease or condition selected from idiopathic pulmonary fibrosis, neurofibromatosis, Hermansky-Pudlak syndrome, diabetic nephropathy, renal failure, hypertrophic cardiomyopathy (HCM), focal segmental glomerulosclerosis (FSGS), radiation-induced fibrosis, multiple sclerosis, and uterine leiomyomas (fibroids) in a patient in need thereof.

In another particular embodiment, the method of the invention is used to treat renal fibrosis, hepatic fibrosis, uterine leiomyomas, keloid scarring, multiple sclerosis, radiation-associated fibrosis, organ transplant rejection, or cancer in a patient in need thereof.

In still another particular embodiment, the method of this invention is used to treat idiopathic pulmonary fibrosis in a patient in need thereof.

In another particular embodiment, the method of this invention is used to treat secondary progressive multiple sclerosis in a patient in need thereof.

In another particular embodiment, the method of this invention is used to treat pancreatic cancer in a patient in need thereof.

In another more particular embodiment, the method of this invention is used to treat renal fibrosis in a patient in need thereof. More particularly the method is used to treat renal fibrosis as the result of diabetic nephropathy, glomerulopathy/FSGS or hypertension-related nephropathy.

Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with pirfenidone. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Therapeutic Kits

The present invention provides kits for use to treat idiopathic pulmonary fibrosis, neurofibromatosis, Hermansky-Pudlak syndrome, diabetic nephropathy, renal fibrosis, hepatic fibrosis, keloid scarring, hypertrophic cardiomyopathy (HCM), glomerulosclerosis (FSGS), radiation-induced fibrosis, multiple sclerosis, organ rejection, cancer, and uterine leiomyomas (fibroids). These kits comprise (a) a pharmaceutical composition comprising a compound of Formula I or a salt, hydrate, or solvate thereof, wherein said pharmaceutical composition is in a container; and (b) instructions describing a method of using the pharmaceutical composition to treat one or more of the aforementioned disease or conditions.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

The invention now being generally described, it will be more readily understood by reference to the following examples which are included merely for purposes of illustration of certain aspects and embodiments of the present invention, and are not intended to limit the invention in any way.

### Determination of Metabolic Stability.

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: Human liver microsomes (20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability: 7.5 mM stock solutions of test compounds are prepared in DMSO. The 7.5 mM stock solutions are diluted to 50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. 10 μL of the 50 μM test compound is added to the microsomes and the mixture is pre-warmed for 10 minutes. Reactions are initiated by addition of pre-warmed NADPH solution. The final reaction volume is 0.5 mL and contains 1 mg/mL human liver microsomes, 1 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contained 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer.

Data analysis: The in vitro half-lives (t_{1/2}s) for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data analysis is performed using Microsoft Excel Software.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I in 100 mM potassium phosphate buffer (pH 7.4) is incubated at 37° C. in triplicate. Positive controls contain 1 μM of pirfenidone instead of a compound of Formula I or II. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

### EXAMPLES

### Example 1

### 5-(Methoxy-d₃-methyl-d₂)-1-phenylpyridin-2(1H)-one (103)

Compound 103 was prepared according to Scheme 6 below. Details of the synthesis are set forth below.

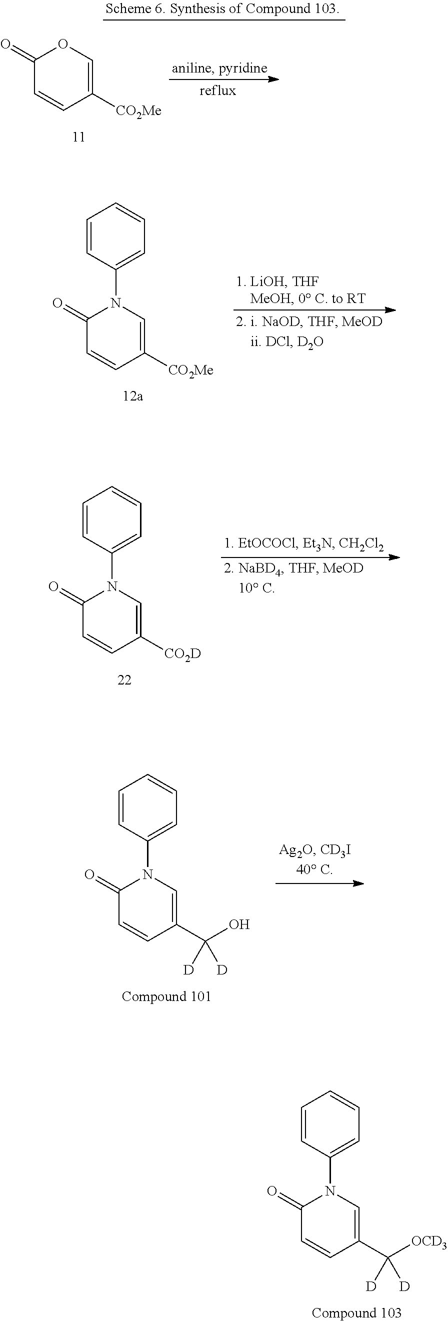

### Step 1. Methyl 6-oxo-1-phenyl-1,6-dihydropyridine-3-carboxylate (12a)

To a round-bottom flask was added methyl 2-oxo-2H-pyran-5-carboxylate 11 (1.54 g, 10.0 mmol), pyridine (30 mL), and aniline (1.82 mL, 20.0 mmol). The resulting mixture was heated at reflux for 6 hours (h). Upon cooling to room temperature (rt), the reaction was diluted with EtOAc and washed with 1N HCl (3 times), saturated aqueous NaHCO₃ (1 time), dried (Na₂SO₄), filtered, and concentrated in vacuo. Purification via column chromatography on an ISCO instrument (0% to 40% EtOAc in hexane) provided 917 mg of the title compound 12a. NMR (CDCl₃): δ 8.24 (m, 1H), 7.92 (ddd, J=0.6, 2.6, 9.6, 1H), 7.56-7.44 (m, 3H), 7.40-7.36 (m, 2H), 6.64 (d, J=9.6, 1H), 3.86 (s, 3H). MS (M+H): 230.1.

### Step 2. 6-Oxo-1-phenyl-1,6-dihydropyridine-3-deuterocarboxylic acid (22)

A round-bottom flask was charged with methyl 6-oxo-1-phenyl-1,6-dihydropyridine-3-carboxylate 12a (2.26 g, 9.87 mmol), THF (58.5 mL) and MeOH (14.6 mL). The resulting slurry was cooled to 0° C. A solution of LiOH (700 mg, 29.2 mmol) in water (29.2 mL) was added dropwise via cannula. After stirring for 5 minutes the ice bath was removed. The mixture was stirred at rt for 45 minutes and then at 35° C. for 30 minutes. After cooling to rt, the mixture was acidified to pH 1 with 1N HCl and extracted with EtOAc (3 times). The combined organic layers were washed with brine, dried (Na₂SO₄), filtered, and concentrated in vacuo to afford 1.95 g of a brown solid which was used without further purification.

A round-bottom flask was charged with 1.30 g of the brown solid, THF (35.8 mL) and MeOD (8.94 mL). A solution of NaOD (99.5 atom % D, 1.86 mL, 40 wt. % in D₂O) was added. After stirring at rt for 1.5 h, the mixture was cooled to 0° C., acidified to pH 1 with DCl (35 wt. % in D₂O), and extracted with EtOAc (3 times). The combined organic layers were dried (Na₂SO₄), filtered and concentrated in vacuo to afford 1.31 g (100%) of the title compound 22 as a brown solid. ¹H NMR (DMSO-d₆): δ 8.22 (d, J=2.4, 1H), 7.91 (dd, J=2.7, 9.1, 1H), 7.62-7.48 (m, 5H), 6.58 (d, J=9.44, 1H). MS (M+H): 216.1.

### Step 3. 5-(Hydroxy(methyl-d₂))-1-phenylpyridin-2(1H)-one (Compound 101)

A round-bottom flask was charged with 6-oxo-1-phenyl-1,6-dihydropyridine-3-deuterocarboxylic acid 22 (476 mg, 2.20 mmol), CH₂Cl₂ (6.11 mL), triethylamine (0.613 mL, 4.40 mmol) and ethyl chloroformate (0.419 mL, 4.40 mmol). The mixture was stirred overnight at rt and then filtered through Celite. The flask and the filter cake were rinsed with THF. The filtered solution was concentrated under reduced pressure then placed under high vacuum for approximately 20 minutes to afford 635 mg of crude carbonic anhydride. This material was dissolved in THF (14.6 mL), cooled to 10° C. and NaBD₄ (293 mg, 7.00 mmol) was added. To the resulting slurry was added MeOD (1.41 mL) via syringe pump at a rate of 0.015 mL/min. No reaction was observed by TLC analysis after 2 h. NaBD₄ (98 atom % D, Cambridge Isotopes Laboratory) (293 mg) was then added, but no reaction was observed. Additional NaBD₄ (586 mg) was added, and after 30 minutes (min), the reaction was determined to be complete by TLC analysis. The reaction was diluted with MeOD and concentrated to near dryness on a rotary evaporator. This process was repeated twice. The resulting solid was suspended in CH₂Cl₂ and silica gel was added. The resulting slurry was concentrated to dryness at reduced pressure, and the remaining solid was added to the top of a silica gel column. Two purifications via column chromatography on an ISCO instrument (0% to 100% EtOAc in hexane) provided 125 mg (28%) of Compound 101, ¹H NMR (CDCl₃): δ 7.52-7.19 (m, 7H), 6.62 (dd, J=0.6, 9.4, 1H), 2.84 (s, 1H). MS (M+H): 204.2.

### Step 4. 5-(methoxy-d₃-methyl-d₂)-1-phenylpyridin-2(1H)-one (Compound 103)

A vial was charged with 5-(hydroxy(methyl-d₂))-1-phenylpyridin-2(1H)-one 101 (59.1 mg, 0.291 mmol), CD₃I (99.5% atom % D, Isotec) (0.418 mL) and Ag₂O (337 mg). The vial was sealed, heated at 40° C. with stirring for 4.5 h and then was cooled to rt. The mixture was filtered through Celite and the filter cake was rinsed with acetonitrile. The filtered solution was concentrated at reduced pressure. Purification via column chromatography on an ISCO instrument (0% to 100% EtOAc in hexane) provided 36.7 mg (57%) of Compound 103. ¹H NMR (CDCl₃): δ 7.53-7.45 (m, 2H), 7.45-7.34 (m, 4H), 7.32 (d, J=2.0, 1H). MS (M+H): 221.2.

### Example 2

### 4-(Methyl-d₃)phenyl-d₄)-5-(hydroxyl(methyl-d₂))-pyridin-2(1H)-one (151)

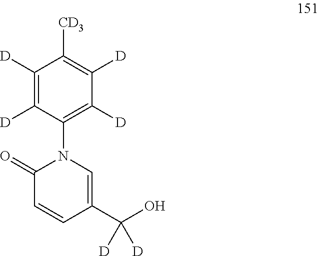

Compound 151 was synthesized in the same manner as Compound 101 (cf. Example 1, Scheme 6, steps 1-3), with the exception that D p-toluidine-d₉ (98 atom % D, CDN Isotopes) was used in place of aniline in step 1, as provided below.

### Step 1. Methyl 1-(4-(methyl-d₃)phenyl-d₄)-6-oxo-1,6-dihydropyridine-3-carboxylate (23)

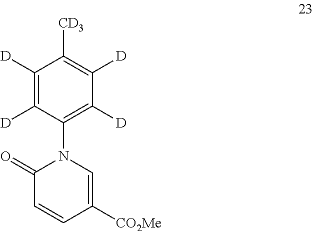

To a round-bottom flask was added methyl 2-oxo-2H-pyran-5-carboxylate 11 (cf. Scheme 6) 2.65 g, 17.2 mmol), pyridine (52.1 mL) and D p-toluidine-d₉ (98 atom % D, CDN Isotopes) (3.0 g, 25.8 mmol). The mixture was heated at reflux for 6 h. Upon cooling to rt, the reaction was diluted with EtOAc and washed with 1N HCl (3 times), saturated aqueous NaHCO₃ (1 time), dried (Na₂SO₄), filtered and concentrated in vacuo. Purification via column chromatography on an ISCO instrument (0% to 40% EtOAc in hexane) provided 2.11 g of 23. NMR (CDCl₃): δ 8.22 (d, J=2.13, 1H), 7.91 (dd, J=2.3, 10.1, 1H), 6.63 (d, J=9.6, 1H), 3.86 (s, 3H). MS (M+H): 251.2.

### Step 2. 1-(4-(Methyl-d₃)phenyl-d₄)-6-oxo-1,6-dihydropyridine-3-carboxylic acid (24)

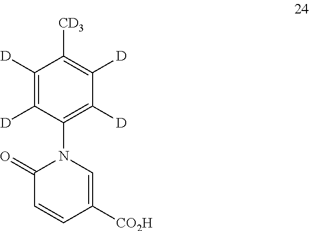

A round-bottom flask was charged with methyl 1-(4-(methyl-d₃)phenyl-d₄)-6-oxo-1,6-dihydropyridine-3-carboxylate 23 (2.11 g, 8.45 mmol), THF (50.0 mL) and MeOH (12.6 mL). The resulting slurry was cooled to 0° C. A solution of LiOH (602 mg, 25.1 mmol) in water (25.1 mL) was added dropwise via cannula. After stirring for 5 min, the ice bath was removed. The mixture was stirred at rt for 45 min and then at 35° C. for 30 min. After cooling to rt, the mixture was acidified to pH 1 with 1N HCl and extracted with EtOAc (3 times). The combined organic layers were washed with brine, dried (Na₂SO₄), filtered and concentrated in vacuo to afford 1.82 g of 24 as a brown solid. MS (M+H): 237.1.

### Step 3. 1-(4-(Methyl-d₃)phenyl-d₄)-5-(hydroxy)(methyl-d₂))-pyridin-2(1H)-one (Compound 151)

A round-bottom flask was charged with 1-(4-(methyl-d₃)phenyl-d₄)-6-oxo-1,6-dihydropyridine-3-carboxylic acid 24 (1.72 g, 6.88 mmol), CH₂Cl₂ (19.1 mL), triethylamine (1.44 mL, 10.4 mmol), cooled to 0° C. and ethyl chloroformate (1.12 L, 10.4 mmol) was added. The mixture was stirred overnight at rt, then filtered through Celite. The flask and the filter cake were rinsed with Et₂O. The filtered solution was concentrated under reduced pressure and the residue placed under high vacuum for approximately 20 min. The resulting material was dissolved in THF (46 mL), cooled to 10° C. and NaBD₄ (98 atom % D, Cambridge Isotope Laboratories) (863 mg, 20.6 mmol) was added. To the resulting slurry was added MeOD (4.15 mL) via syringe pump at a rate of 0.034 mL/min. When the addition was complete, the reaction was diluted with MeOD and concentrated to near dryness on a rotary evaporator. This process was repeated twice. The resulting solid was suspended in CH₂Cl₂, silica gel was added, the slurry concentrated to dryness in vacuo, and the resulting solid was added to the top of a silica gel column. Two purifications via column chromatography on an ISCO instrument (0% to 10% MeOH in CH₂Cl₂, followed by a second column with 50 to 100% EtOAc in hexanes) provided 176 mg (11%) of the title compound 151. ¹H NMR (CDCl₃): δ 7.42 (dd, J=2.5, 9.6, 1H), 7.31 (d, J=2.4, 1H), 6.64 (d, J=9.6, 1H), 2.41 (s, 1H). MS (M+H) 225.3.

### Example 3

### 5-(Hydroxy(methyl-d₂))-1-(phenyl-d₅)pyridin-2(1H)-one (131)

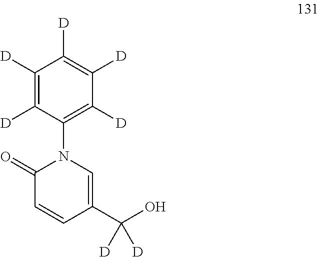

Compound 131 was synthesized in the same manner as Compound 151 with the exception that 2,3,4,5,6-d₅-aniline (98% atom % D, CDN Isotopes) was used in place of D p-toluidine-d₉ in step 1, as provided below.

### Step 1. Methyl 6-oxo-1-(phenyl-d₅)-1,6-dihydropyridine-3-carboxylate (25)

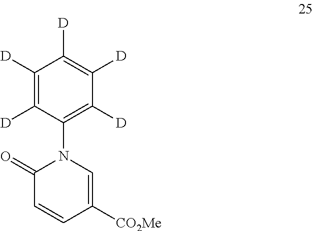

To a round-bottom flask was added methyl 2-oxo-2H-pyran-5-carboxylate 11 (3.14 g, 20.4 mmol), pyridine (61.8 mL) and 2,3,4,5,6-d₅-aniline (98% atom % D, CDN Isotopes) (3.0 g, 30.6 mmol). The mixture was heated at reflux for 16 h. Upon cooling to rt, the reaction was diluted with EtOAc and washed with 1N HCl (3 times), saturated aqueous NaHCO₃ (1 time), dried (Na₂SO₄), filtered and concentrated in vacuo. Purification via column chromatography on an ISCO instrument (0% to 40% EtOAc in hexane) provided 1.85 g of the desired product 25. ¹H NMR (CDCl₃): δ 8.24 (d, J=2.4, 1H), 7.92 (dd, J=2.6, 9.6, 1H), 6.64 (d, J=9.7, 1H), 3.86 (s, 3H). MS (M+H): 235.1.

### Step 2. 6-Oxo-1-(phenyl-d₅)-1,6-dihydropyridine-3-carboxylic acid (26)

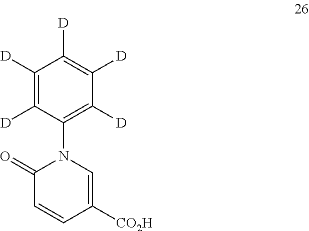

A round-bottom flask was charged with methyl 6-oxo-1-(phenyl-d₅)-1,6-dihydropyridine-3-carboxylate 25 (1.85 mg, 7.88 mmol), THF (46.6 mL) and MeOH (11.8 mL). The resulting slurry was cooled to 0° C. A solution of LiOH (562 mg, 23.4 mmol) in water (23.4 mL) was added dropwise via cannula. After stirring for 5 min the ice bath was removed. The mixture was stirred at rt for 45 min. The mixture was acidified to pH 1 with 1N HCl and extracted with EtOAc (3 times). The combined organic layers were washed with brine, dried (Na₂SO₄), filtered and concentrated in vacuo. Purification via column chromatography on an ISCO instrument (0% to 30% MeOH in dichloromethane) provided 851 mg of 26. ¹H NMR (DMSO-d₆): δ 12.9 (br s, 1H), 8.11 (dd, J=0.5, 2.4, 1H), 7.82 (dd, J=2.7, 9.4, 1H), 6.48 (d, J=9.6, 1H). MS (M+H): 221.2.

### Step 3. 5-(Hydroxy(methyl-d₂))-1-(phenyl-d₅)pyridin-2(1H)-one (Compound 131)

A round-bottom flask was charged with 6-oxo-1-(phenyl-d₅)-1,6-dihydropyridine-3-carboxylic acid 26 (793 mg, 3.60 mmol), CH₂Cl₂ (9.99 mL), triethylamine (0.753 mL, 5.42 mmol) and ethyl chloroformate (0.325 mL, 5.42 mmol). The mixture was stirred overnight at rt and then filtered through Celite. The flask and the filter cake were rinsed with Et₂O. The filtered solution was concentrated and the residue placed under high vacuum for approximately 20 min. This material was dissolved in THF (25.5 mL), cooled to 10° C., and NaBD₄ (98 atom % D, Cambridge Isotopes Laboratories) (229 mg, 5.49 mmol) was added. To the resulting slurry was added MeOD (2.30 mL) via syringe pump at a rate of 0.034) mL/min. When the addition was complete, the reaction was diluted with MeOD and concentrated to near dryness on a rotary evaporator. This process was repeated twice. The resulting solid was suspended in CH₂Cl₂, silica gel was added, the slurry concentrated to dryness in vacuo, and the resulting solid was added to the top of a silica gel column. Purification via column chromatography on an ISCO instrument (0% to 10% MeOH in CH₂Cl₂) provided 121 mg of the title compound 131. ¹H NMR (DMSO-d₆): δ 7.31 (dd, J=3.0, 9.7, 1H), 6.98 (d, J=3.0, 1H), 6.42 (d, J=9.7, 1H). MS (M+H): 209.2.

### Example 4

### 1-(4-Fluorophenyl-d₄)-5-(hydroxy(methyl-d₂))pyridin-2(1H)-one (139)

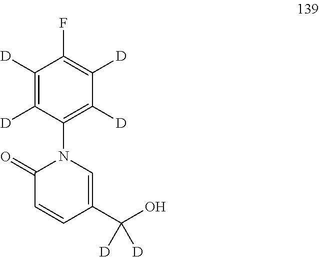

Compound 139 was synthesized in the same manner as Compound 151 with the exception that 4-fluoro-2,3,5,6-d₄-aniline (98% atom % D, CDN Isotopes) was used in place of D p-toluidine-d₉ in step 1. ¹H NMR (CDCl₃): δ 7.44 (dd, J=2.5, 9.6, 1H), 7.31 (d, J=2.5, 1H), 6.67 (d, J=9.3, 1H). MS (M+H): 226.1. After storage for approximately 2 weeks at about −20° C., compound 139 was found to have decomposed.

### Example 5

### 1-(4-Chlorophenyl-d₄)-5-(hydroxy(methyl-d₂))-pyridin-2(1H)-one (161)

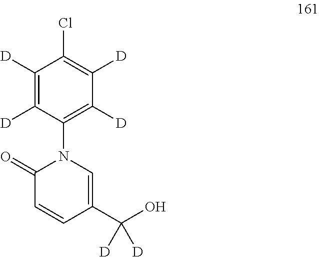

Compound 161 was synthesized in the same manner as Compound 101 (Example 1, Scheme 6, steps 1-3), except that 4-chloro-2,3,5,6-d₄-aniline (98% atom % D, CDN Isotopes) was used in place of aniline in step 1. MS (M+H): 242.1. After storage for approximately 2 weeks at about −20° C., compound 161 was found to have decomposed.

### Example 6

### 5-(Hydroxyl(methyl-d₂))-1-(4-(methoxy-d₃)phenyl-d₄)pyridin-2(1H)-one (156)

Compound 156 was prepared according to Scheme 7 below. Details of the synthesis are set forth below.

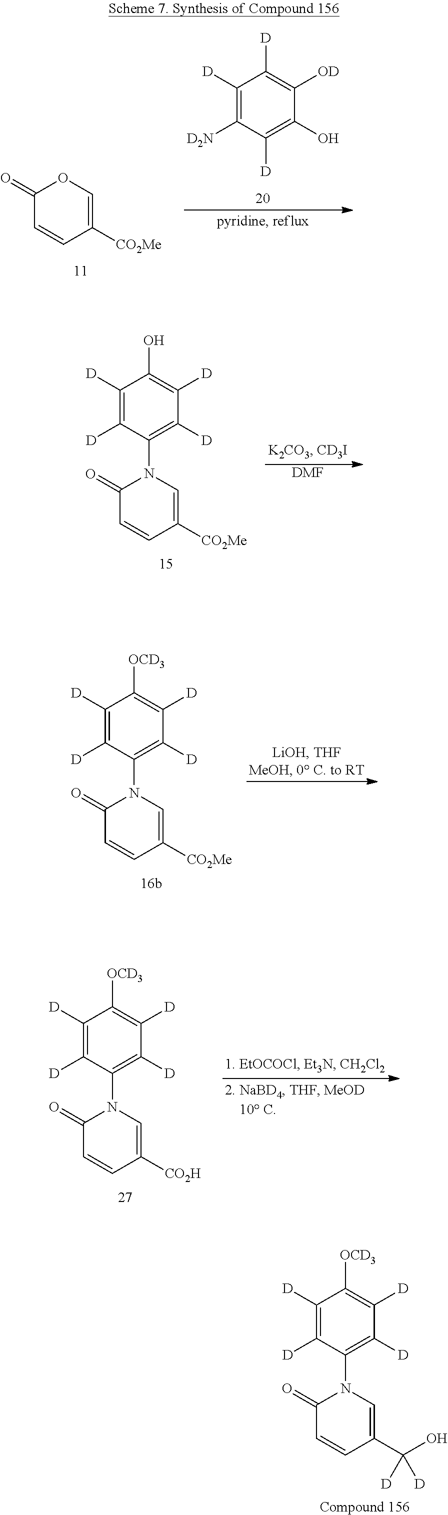

### Step 1. Methyl 1-(4-hydroxyphenyl-d₄)-6-oxo-1,6-dihydropyridine-3-carboxylate (15)

To a solution of 11 (0.177 g, 1.15 mmol) in pyridine (3.48 mL) was added 4-aminophenol-d₇ 20 (97 atom % D, CDN Isotopes) (0.200 g, 1.72 mmol). The mixture was heated at reflux for 6 h. Upon cooling to rt, the reaction was diluted with EtOAc and washed with 1N HCl (3 times), saturated aqueous NaHCO₃ (1 time), dried (Na₂SO₄), filtered and concentrated in vacuo. Purification via column chromatography on an ISCO instrument (0% to 100% EtOAc in hexane) provided 102.1 mg (36%) of compound 15. MS (M+H): 250.1.

### Step 2. Methyl 1-(4-(methoxy-d₃)phenyl-d₄)-6-oxo-1,6-dihydropyridine-3-carboxylate (16b)

To a solution of methyl 1-(4-hydroxyphenyl-d₄)-6-oxo-1,6-dihydropyridine-3-carboxylate 15 (0.092 g, 0.369 mmol) in DMF (0.738 mL) was added K₂CO₃ (0.102 g, 0.738 mmol). After stirring for 10 min, CD₃I (99.5 atom % D, Isotech) (0.046 mL, 0.738 mmol) was added and the mixture was stirred for 4 h. CD₃I (0.092 mL) was then added and stirring continued for 21 h at which time 0.092 mL of CD₃I and 102 mg of K₂CO₃ were added. After stirring for an additional 24 h, the reaction was diluted with EtOAc, washed with H₂O (3×), dried (Na₂SO₄), concentrated in vacuo and filtered. Purification via column chromatography on an ISCO instrument (0% to 50% EtOAc in hexanes) afforded 67 mg (68%) of compound 16b. MS (M+H): 267.1.

### Step 3. 1-(4-(Methoxy-d₃)phenyl-d₄)-6-oxo-1,6-dihydropyridine-3-carboxylic acid (27)

A solution of methyl 1-(4-(methoxy-d₃)phenyl-d₄)-6-oxo-1,6-dihydropyridine-3-carboxylate 16b (1.877 g, 7.05 mmol) in THF (42 mL) and MeOH (10.6 mL) was cooled to 0° C. A solution of LiOH (506 mg, 21.1 mmol) in water was added dropwise via cannula. After stirring for 5 min the ice bath was removed. The mixture was stirred at rt for 45 min, then acidified to pH 1 with 1N HCl and extracted with EtOAc (3×). The combined organic layers were dried (Na₂SO₄), filtered and concentrated in vacuo to dryness. Purification via column chromatography on an ISCO instrument (0% to 10% MeOH in dichloromethane) afforded 1.29 g (72%) of Compound 27. MS (M+H): 253.1.

### Step 4. (Ethyl carbonic) 1-(4-(methoxy-d₃)phenyl-d₄)-6-oxo-1,6-dihydropyridine-3-carboxylic anhydride (27)

To a solution of 1-(4-(methoxy-d₃)phenyl-d₄)-6-oxo-1,6-dihydropyridine-3-carboxylic acid 27 (50 mg, 0.198 mmol) in CH₂Cl₂ (0.549 mL) at −40° C. was added triethylamine (0.041 mL, 0.361 mmol) and ethyl chloroformate (0.032 mL, 0.361 mmol). The mixture was allowed to warm slowly to rt overnight with stirring then was filtered through Celite and the flask and the filter cake were rinsed with Et₂O. The filtered solution was concentrated under reduced pressure and placed under high vacuum for approximately 20 minutes. This procedure was repeated nine times to afford 464 mg of the crude carbonic anhydride which was used without further purification.

### Step 5. 5-(Hydroxylmethyl-d₂))-1-(4-(methoxy-d₃)phenyl-d₄)pyridin-2(1H)-one (Compound 156)

A solution of (ethyl carbonic) 1-(4-(methoxy-d₃)phenyl-d₄)-6-oxo-1,6-dihydropyridine-3-carboxylic anhydride 27 (464 mg, approximately. 1.43 mmol) in THF (14.8 mL) was cooled to 10° C. and NaBD₄ (98 atom % D, Cambridge Isotopes Laboratories) (295 mg, 7.01 mmol) was added. To the resulting slurry was added MeOD (1.42 mL) via syringe pump at a rate of 0.015 mL/min. Upon completion, the reaction was diluted with MeOD and concentrated nearly to dryness on a rotary evaporator. This process was repeated twice. The resulting solid was suspended in CH₂Cl₂, silica gel was added, the slurry concentrated to dryness in vacuo, and the resulting solid was added to the top of a silica gel column. Purification via column chromatography on an ISCO instrument (0 to 10% MeOH in CH₂Cl₂) provided impure final product. A further purification by ISCO (50 to 100% EtOAc in hexanes) followed by a preparatory HPLC purification afforded 116 mg of the final product 156 in approximately 85% purity. ¹H NMR (CDCl₃): δ 7.43 (dd, J=2.7, 9.5, 1H), 7.31 (dd, J=0.6, 2.5, 1H), 6.66 (dd, J=0.6, 9.5, 1H). MS (M+H): 241.1. After storage for approximately 2 weeks at about −20° C., compound 156 was found to have decomposed.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A compound represented by Formula I: 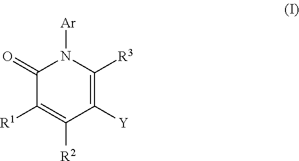
or a pharmaceutically acceptable salt thereof, wherein:
Ar is 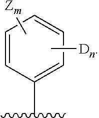
wherein:
Z, if present, is selected from halo, —OH, —CH₃, —CD₃, —OCH₃, —OCD₃, CF₃, and —NO₂;
m is 0 or 1;
n is 0 or an integer from 1 to 5;
m+n≦5;
R¹ is hydrogen, deuterium, or a group selected from phenyl, chlorophenyl, and C₁-C₃ alkyl, which group is optionally substituted with one or more deuterium atoms; each of R² and R³ is independently hydrogen or deuterium; and
Y is selected from —CD₃, —CD₂CD₃, —(CD₂)₂CD₃, —C(O)D, —C(O)CD₃, —CD₂F, —CDF₂, —CD₂OCH₃, —CD₂OCD₃, —CH₂OCD₃, —CD₂OH, and —CF₃;
provided that at least one of R¹, R², R³, Y, Z and Ar is or contains deuterium; and
provided that when R¹ is hydrogen or deuterium, and Y is —CD₃, then m is 1.

2. The compound of claim 1 wherein if Y is CD₂OH and Ar is 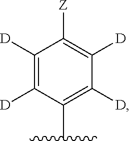
then Z is not —F, —Cl, or —OCD₃.

3. The compound of claim 1, wherein m is 1, the compound having the Formula Ia: 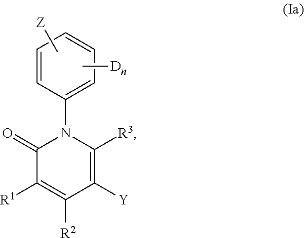
wherein:
n is 0, 1, 2, 3, or 4.

4. The compound of claim 3, wherein n is 0 or 4.

5. The compound of claim 4, wherein Z, if present, is selected from —OH, —CH₃, —CD₃, —CF₃, and —NO₂.

6. The compound of claim 4, having the Formula Ib:

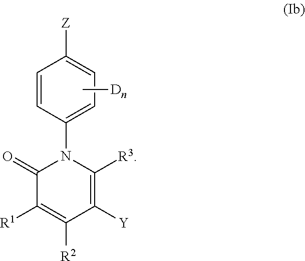

7. The compound of claim 6, wherein R¹ is selected from hydrogen, deuterium, —CH₃, —CD₃, —CD₂CH₃, —CD₂CD₃, and —CH₂CD₃.

8. The compound of claim 6, wherein R¹ is hydrogen or deuterium.

9. The compound of claim 8, wherein each of R¹, R², and R³ is hydrogen and Y is selected from —CF₃—CD₃, —CD₂CD₃, CD₂)₂CD₃, —CD₂F, —CDF₂, —CD₂OCH₃, —CD₂OCD₃, —CH₂OCD₃, and —CD₂OH.

10. (canceled)

11. A compound represented by Formula II: 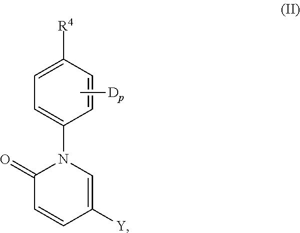
wherein:
Y is selected from —CF₃, —CD₃, —CD₂CD₃, —(CD₂)₂CD₃, —CD₂F, —CDF₂, —CD₂OCH₃, —CD₂OCD₃, —CH₂OCD₃, and —CD₂OH;
R⁴ is selected from hydrogen, deuterium, fluorine, chlorine, —OH, —CH₃, —CD₃, —OCH₃, —OCD₃, or —CF₃;
p is 0 or an integer from 1 to 4;
when Y is —CF₃, R⁴ is selected from —CD₃, and —OCD₃; and
when Y is —CD₃, R⁴ is not hydrogen or deuterium.

12. The compound of claim 11, selected from any one of the compounds set forth in the table below, wherein p is 0:

| Compound | R⁴ | Y |
|---|---|---|
| 101 | H | —CD₂OH |
| 102 | H | —CD₂OCH₃ |
| 103 | H | —CD₂OCD₃ |
| 104 | OH | —CD₃ |
| 105 | OH | —CD₂OH |
| 106 | OH | —CD₂OCH₃ |
| 107 | OH | —CD₂OCD₃ |
| 108 | F | —CD₃ |
| 109 | F | —CD₂OH |
| 110 | F | —CD₂OCH₃ |
| 111 | F | —CD₂OCD₃ |
| 112 | CH₃ | —CD₃ |
| 113 | CH₃ | —CD₂OH |
| 114 | CH₃ | —CD₂OCH₃ |
| 115 | CH₃ | —CD₂OCD₃ |
| 116 | OCH₃ | —CD₃ |
| 117 | OCH₃ | —CD₂OH |
| 118 | OCH₃ | —CD₂OCH₃ |
| 119 | OCH₃ | —CD₂OCD₃ |
| 120 | CD₃ | —CD₃ |
| 121 | CD₃ | —CD₂OH |
| 122 | CD₃ | —CD₂OCH₃ |
| 123 | CD₃ | —CD₂OCD₃ |
| 124 | CD₃ | —CF₃ |
| 125 | OCD₃ | —CD₃ |
| 126 | OCD₃ | —CD₂OH |
| 127 | OCD₃ | —CD₂OCH₃ |
| 128 | OCD₃ | —CD₂OCD₃ |
| 129 | OCD₃ | —CF₃. |

13. The compound of claim 11, wherein R⁴ is selected from hydrogen, deuterium, —OH, —CH₃, —CD₃, and —CF₃.

14. The compound of claim 13, wherein the compound is selected from the group consisting of the compounds set forth in the table below, wherein p is 0:

| Compound | R⁴ | Y |
|---|---|---|
| 101 | H | —CD₂OH |
| 102 | H | —CD₂OCH₃ |
| 103 | H | —CD₂OCD₃ |
| 104 | OH | —CD₃ |
| 105 | OH | —CD₂OH |
| 106 | OH | —CD₂OCH₃ |
| 107 | OH | —CD₂OCD₃ |
| 112 | CH₃ | —CD₃ |
| 113 | CH₃ | —CD₂OH |
| 114 | CH₃ | —CD₂OCH₃ |
| 115 | CH₃ | —CD₂OCD₃ |
| 120 | CD₃ | —CD₃ |
| 121 | CD₃ | —CD₂OH |
| 122 | CD₃ | —CD₂OCH₃ |
| 123 | CD₃ | —CD₂OCD₃ |
| 124 | CD₃ | —CF₃. |

15. The compound of claim 11, represented by Formula IIa:

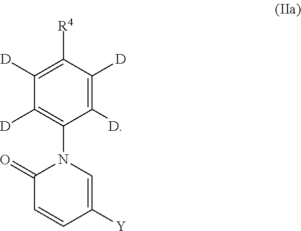

16. The compound of claim 15, selected from any one of the compounds set forth in the table below:

| Compound | R⁴ | Y |
|---|---|---|
| 131 | D | —CD₂OH |
| 132 | D | —CD₂OCH₃ |
| 133 | D | —CD₂OCD₃ |
| 134 | OH | —CD₃ |
| 135 | OH | —CD₂OH |
| 136 | OH | —CD₂OCH₃ |
| 137 | OH | —CD₂OCD₃ |
| 138 | F | —CD₃ |
| 139 | F | —CD₂OH |
| 140 | F | —CD₂OCH₃ |
| 141 | F | —CD₂OCD₃ |
| 142 | CH₃ | —CD₃ |
| 143 | CH₃ | —CD₂OH |
| 144 | CH₃ | —CD₂OCH₃ |
| 145 | CH₃ | —CD₂OCD₃ |
| 146 | OCH₃ | —CD₃ |
| 147 | OCH₃ | —CD₂OH |
| 148 | OCH₃ | —CD₂OCH₃ |
| 149 | OCH₃ | —CD₂OCD₃ |
| 150 | CD₃ | —CD₃ |
| 151 | CD₃ | —CD₂OH |
| 152 | CD₃ | —CD₂OCH₃ |
| 153 | CD₃ | —CD₂OCD₃ |
| 154 | CD₃ | —CF₃ |
| 155 | OCD₃ | —CD₃ |
| 156 | OCD₃ | —CD₂OH |
| 157 | OCD₃ | —CD₂OCH₃ |
| 158 | OCD₃ | —CD₂OCD₃ |
| 159 | OCD₃ | —CF₃ |
| 160 | Cl | —CD₃ |
| 161 | Cl | —CD₂OH |
| 162 | Cl | —CD₂OCH₃ |
| 163 | Cl | —CD₂OCD₃. |

17. The compound of claim 15, wherein R⁴ is selected from hydrogen, deuterium, —OH, —CH₃, —CD₃, and —CF₃.

18. The compound of claim 17, wherein the compound is selected from the group consisting of

| Compound | R⁴ | Y |
|---|---|---|
| 131 | D | —CD₂OH |
| 132 | D | —CD₂OCH₃ |
| 133 | D | —CD₂OCD₃ |
| 134 | OH | —CD₃ |
| 135 | OH | —CD₂OH |
| 136 | OH | —CD₂OCH₃ |
| 137 | OH | —CD₂OCD₃ |
| 142 | CH₃ | —CD₃ |
| 143 | CH₃ | —CD₂OH |
| 144 | CH₃ | —CD₂OCH₃ |
| 145 | CH₃ | —CD₂OCD₃ |
| 150 | CD₃ | —CD₃ |
| 151 | CD₃ | —CD₂OH |
| 152 | CD₃ | —CD₂OCH₃ |
| 153 | CD₃ | —CD₂OCD₃ |
| 154 | CD₃ | —CF₃. |

19. The compound of claim 1, wherein any atom not designated as deuterium is present at its natural isotopic abundance.

20. A pyrogen-free pharmaceutical composition comprising a compound of claim 1 and a pharmaceutically acceptable carrier.

21. (canceled)

22. A method of treating a disease selected from idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; focal segmental glomerulosclerosis (FSGS); radiation-induced fibrosis; multiple sclerosis; secondary progressive multiple sclerosis; uterine leiomyomas (fibroids); alcoholic liver disease including hepatic steatosis, hepatic fibrosis and hepatic cirrhosis; keloid scarring; hepatitis C virus (HCV) infection; proliferative disorders; angiogenesis-mediated disorders; cancer; fibrotic disorders; interstitial lung diseases; atrial fibrillation (AF); organ transplant rejection; and fibrous skin diseases in a patient in need thereof comprising the step of administering to the patient an effective amount of the composition of claim 20.

23. The method of claim 22, wherein the disease or condition is selected from renal fibrosis, hepatic fibrosis, uterine leiomyomas, keloid scarring, secondary progressive multiple sclerosis, radiation-associated fibrosis, organ transplant rejection, and pancreatic cancer.

24. The method of claim 23, wherein the disease is renal fibrosis.

25. (canceled)

26. (canceled)
